(19) United States
(12) Patent Application Publication
(10) Pub. No.: US 2001/0036645 A1
(43) Pub. Date: Nov. 1, 2001

(54) Analyte detector and analyte detection method

(76) Inventors: John C. McNeirney, Fairburn, GA (US); William H. Burns JR., Orchard Park, NY (US); Roberta Burns, Orchard Park, NY (US)

Correspondence Address: R. Kent Roberts, Hodgson Russ LLP, Suite 2000, One M&T Plaza, Buffalo, NY, 14203-2391, US

(21) Appl. No.: 09/817,909

(22) Filed: Mar. 26, 2001

Related U.S. Application Data
(63) Continuation-in-part of application No. 09/408,215, filed on Sep. 29, 1999, now abandoned.
(60) Provisional application No. 60/122,658, filed on Mar. 3, 1999.
(60) Provisional application No. 60/102,199, filed on Sep. 29, 1998.

(51) Int. Cl. G01N 33/53; G01N 33/537; G01N 33/543; C12M 1/34

(52) U.S. Cl. 435/7.92; 435/287.2

(57) ABSTRACT

The invention relates to an analyte detector and an analyte detection method. An analyte detector according to the invention may include a first variable volume reagent container and a sample receiving cup in fluid communication with the first variable volume reagent container. The detector may include a capture chamber in fluid communication with the first variable volume reagent container and the sample receiving cup. The detector may include a first antibody residing in the capture chamber, the first antibody being specific for an analyte of interest. A method according to the invention involves using a detector such as that described above.

### CROSS-REFERENCE TO RELATED APPLICATIONS

This is a continuation-in-part of U.S. patent application Ser. No. 09/408,215 (filed Sep. 29, 1999), and applicants claim priority to that patent application, and that patent application is incorporated herein by reference. Applicants also claim priority to two earlier filed U.S. provisional patent applications, Ser. No. 60/122,658 (filed Mar. 3, 1999) and No. 60/102,199 (filed Sep. 29, 1998), both of which are incorporated herein by reference.

### FIELD OF THE INVENTION

The invention relates to an analyte detector and an analyte detection method. The invention may be implemented so as to be portable. The invention may be adapted to detect when a mammal will or has ovulated, and the description that follows will be directed from time to time to that embodiment for purposes of illustrating the invention. However, the invention is not limited to detecting when a mammal will or has ovulated. In particular, the invention may be directed to determining the onset of ovulation based upon the detection of hormones in a blood sample of a mammal.

### BACKGROUND OF THE INVENTION

Known analyte detectors are available in two general categories, solid phase and liquid phase. Solid phase detectors depend on capillary action to move a sample through a fibrous matrix material to one or more sensing zones. The sensing zones contain reactants bound to the matrix material. The presence of the analyte is generally required for production of and/or persistence of a detectable substance. The detectable substance may be fluorescent, chemiluminescent or radioactive. The amount of detectable substance produced may be correlated to a standard to determine how much of the analyte was in the sample.

Such solid phase detectors have shortcomings. First, precise measurement of the amount of sample reacted and transported to the sensing zone is difficult, if not impossible. Second, channeling in the fibrous matrix material results in only partial completion of the reaction between the analyte and the reactants. These first two shortcomings result in solid phase detectors usually being used only for qualitative or semi-quantitative analysis, but not quantitative analysis. Third, the types of reactants that can properly bind to the matrix material are limited, and are typically complex organic compounds.

Liquid phase detectors have many of the same shortcomings as solid phase detectors. In addition, liquid phase detectors are typically limited to a single reactant, and are therefore limited in the types of analytes that can be detected. Some known liquid phase detectors have more than one reactant, but these require the user to add various reactants in a specified sequence at specified times. Such liquid phase detectors are difficult to use and prone to producing erroneous results.

Analyte detectors of the solid phase type and the liquid phase type are available for determining when a female mammal has ovulated. The analytes of relevance in the ovulation process include luteinizing hormone (“LH”), follicle-stimulating hormone (“FSH”), progesterone, estradiol, and estrogen. Concentrations of these hormones fluctuate throughout the female sex cycle. Hormone peaks and troughs are detectable within the blood, thus providing potential for ovulation prediction.

Of these hormones, LH has the most pronounced surge. Immediately before ovulation across all known mammalian species, LH levels rise significantly. In almost all mammals, LH surges within a short, confined period before ovulation occurs. Therefore, LH-blood concentration is a reliable indicator for ensuing ovulation.

FSH also varies its concentrations before ovulation. However, the rise in FSH concentration is slight and gradual. Therefore, precise species specific mechanisms are important to detect changes in FSH concentration. Additionally, FSH is not present in detectable levels in some mammals.

The LH surge causes the granulosa cells of the ovary to begin progesterone production. Progesterone levels generally increase after ovulation. Like FSH, progesterone exhibits moderate concentration differences, accentuated by a steady rise and fall. Progesterone production and secretion is stimulated by changing LH concentrations.

Another hormone essential for the female sex cycle is estrogen. Like the previously described hormones, estrogen experiences a rise and fall during the ovulatory process of a mammalian female. Some estrogen increases are initiated during the pre-ovulatory antral expansion of the ovum; during this portion of the cycle, increasing FSH blood concentrations cause estrogen levels to also rise. In humans, this rise occurs several days prior to the ovum's release, but before the LH surge. However, in cows estrogen levels rise about a week after ovulation. In contrast, the steady increase of the production of progesterone in primates and pigs is the cause of estrogen increases in these animals. Therefore, the point at which estrogen peaks during the female sex cycle is nearly species specific.

Other methods for ovulation detection include a blood serum radioimmunoassay analysis, as first described by Sakai et al., “Evidence For Alterations in Luteinizing Hormone Secreted in Rhesus Monkeys with Normal and Inadequate Luten Phases Using Radioreceptor and Radioimmunoassay,” Endocrinology, 104:1217-1225 (1979). However, this radioimmunoassay measures both beta subunits and LH hormones in the serum, therefore, it is not a reliable LH indicator. Selvaraj et al., “Development of an LH Receptor Assay Capable of Measuring Serum LH/CG in a Wide Variety of Species,” J. Reproduction & Fertility, 98: 611-616 (1993) developed a radioreceptor assay measuring only LH-blood serum concentrations. This assay produced reliable results and was more cost effective, although the assay still required either a homogenized blood or urine serum for testing.

The most reliable test to determine the ovulation time period of a mammal is a clinical analysis of a blood sample. However, this procedure is expensive because it requires the assistance of a trained clinician and the drawn blood sample must be processed in a laboratory. Furthermore, because of the processing delay clinical testing requires, the predictive ovulation window is reduced, decreasing the probability of impregnation. An additional complication associated with this form of testing occurs if the female is not experiencing an LH surge at the time of the blood test. Variables including irregular cycling, a condition known as oligomenorrhoea, will prevent a mammalian female from ovulating during an anticipated time frame. Therefore, testing must be repeated until ovulation is detected, potentially incurring considerable expenses.

### SUMMARY OF THE INVENTION

The invention relates to an analyte detector and an analyte detection method. An analyte detector according to the invention may include a first variable volume reagent container and a sample receiving cup in fluid communication with the first variable volume reagent container. The detector may include a capture chamber in fluid communication with the first variable volume reagent container and the sample receiving cup. The detector may include a first antibody residing in the capture chamber, the first antibody being specific for an analyte of interest.

An analyte detection method according to the invention may include providing an analyte detector, such as that described above, and providing a sample of body fluid in the sample receiving cup. The volume of the first variable volume reagent container is reduced to force the first reagent toward the capture chamber. Then a determination is made as to whether the first antibody combined with the analyte of interest and how much analyte was in the sample.

By using the detector and detection method of the invention, fast, reliable, and inexpensive testing is provided to determine whether a female mammal is ready for breeding. Thus, mammal breeders can more accurately determine when to mate the mammal. In the pig breeding industry, for example, this should result in savings of up to hundreds of millions of dollars per year.

### BRIEF DESCRIPTION OF THE DRAWINGS

FIG. 1 is a schematic plan view of a detector according to the invention;

FIG. 2 is a cross-sectioned side view of a portion of the detector depicted in FIG. 1;

FIG. 3 is a plan view of the sample receiving cup shown cross-sectioned in FIG. 2;

FIG. 4 is a side view of a plug according to the present invention; and

FIG. 5 is a flow diagram of a method according to the invention.

### DETAILED DESCRIPTION OF THE INVENTION

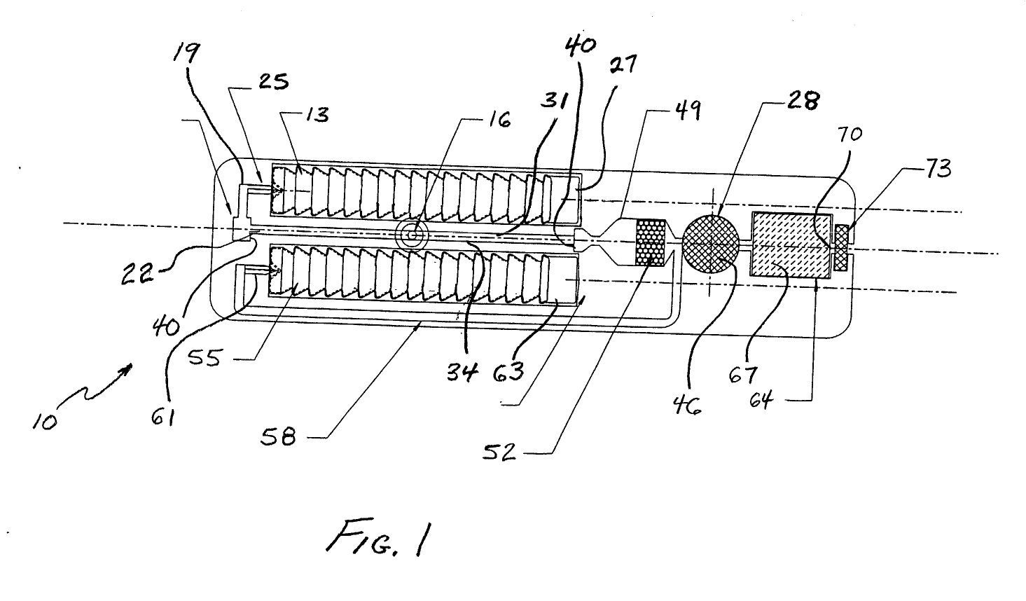

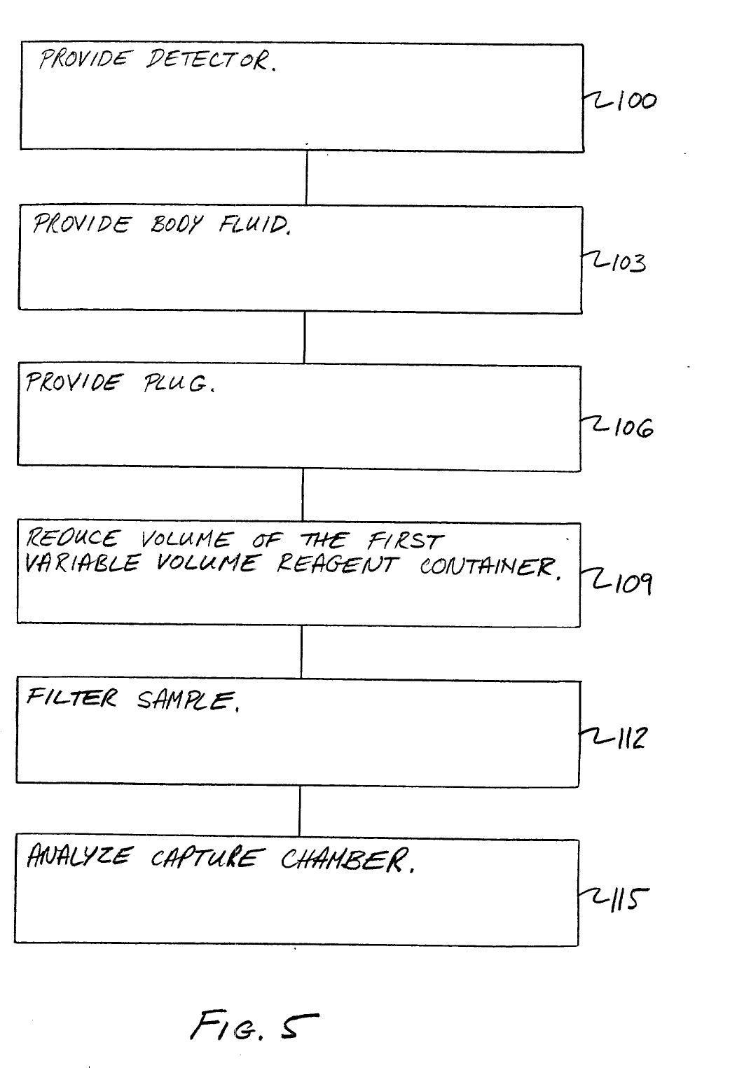

A detector 10 according to the invention is shown in FIGS. 1 through 4. Such a detector 10 includes a first variable volume reagent container 13, and a sample receiving cup 16 in fluid communication with the first variable volume reagent container 13. The first variable volume reagent container 13 may have therein a first reagent, such as a buffer solution. The first reagent may also include an antibody specific to the analyte of interest, or specific to a combination of the analyte of interest and an antibody.

To provide fluid communication between the first variable volume reagent container 13 and the sample receiving cup 16, the invention may include a first reagent transmission tube 19 joined to or capable of joining to the first variable volume reagent container 13 and joined to the sample receiving cup 16. At least part of the first reagent transmission tube 19 may be a capillary tube 22. An end 25 of the first reagent transmission tube 19 may be beveled to provide a penetrating edge proximate to the first variable volume reagent container 13. The beveled end 25 may be used to pierce the first variable volume reagent container 13 to allow reagent inside to flow from the first variable volume reagent container 13 to the first reagent transmission tube 19. For example, the first variable volume reagent container 13 may be moved toward the beveled end 25 such that a portion of the first variable volume reagent container 13 is pierced by the beveled end 25.

The first variable volume reagent container 13 may be collapsible. The first variable volume reagent container 13 may be made from polyethylene, polypropylene, or similar polymers, and may have wall thicknesses small enough to facilitate the collapsible feature. A first plunger 27 may be used to collapse the first variable volume reagent container 13 toward the end 25.

The embodiment of the invention shown in FIGS. 1 through 4 includes a capture chamber 28 in fluid communication with the first variable volume reagent container 13 and the sample receiving cup 16. To provide fluid communication between the capture chamber 28 and the sample receiving cup 16, a sample transmission tube 31 may be joined to or capable of joining to the sample receiving cup 16 and joined to the capture chamber 28. At least part of the sample transmission tube 31 may be a capillary tube 34.

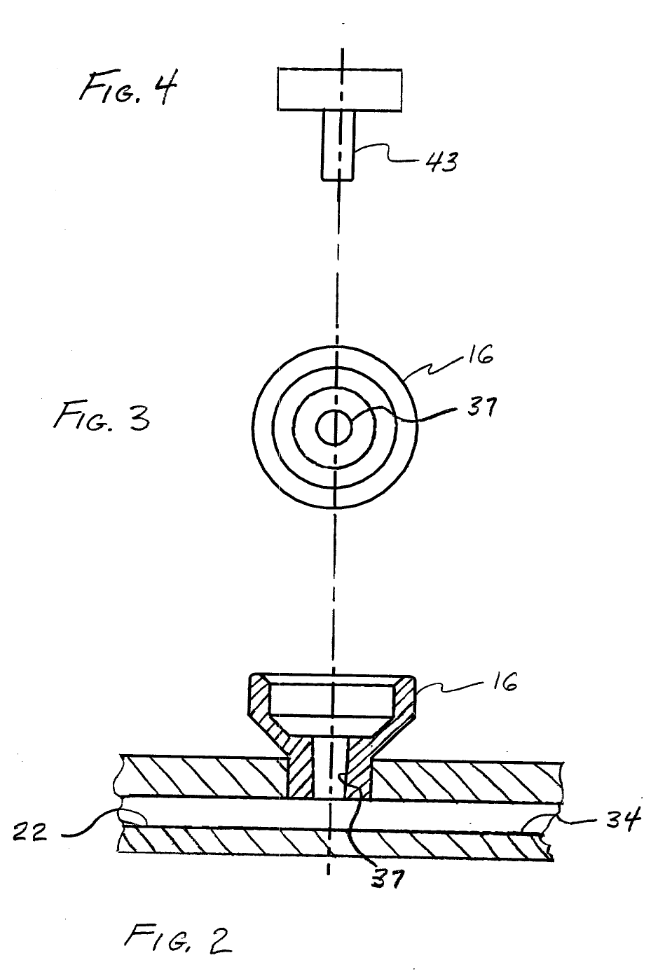

As shown in FIG. 2, a drain 37 may be provided in the sample receiving cup 16 that is joined to both the sample transmission tube 31 and the first reagent transmission tube 19. The drain 37 may be positioned such that the sample flows from the sample receiving cup 16, through the drain 37 to the portions of the sample transmission tube 31 and the first reagent transmission tube 19 that are capillary tubes 22, 34. The capillary tubes 22, 34 draw the sample from the sample receiving cup 16 until the capillary tubes 22, 34 are full of the sample. The capillary breaks 40 are locations where the inside diameter of the sample transmission tube 31 and the first reagent transmission tube 19 increase abruptly. The capillary breaks 40 limit the amount of sample that is drawn from the sample receiving cup 16 into the capillary tubes 22, 34. In this manner, a known amount of sample is tested in the detector 10. This feature helps to permit quantitative, as opposed to qualitative, analysis of the sample.

The invention may include a plug 43 sized to be installed in the sample receiving cup 16. Once the sample is placed in the sample receiving cup 16, the plug 43 may be used to push the sample into the first reagent transmission tube 19, or the sample transmission tube 31, or both. In one embodiment of the invention, the plug 43 has a diameter approximately equal to an inside diameter of the drain 37 in order to prevent the sample from being pushed back into the sample receiving cup 16.

Residing in the capture chamber 28 may be a first antibody specific for an analyte of interest. As used herein, “specific” means an antibody which has a high binding affinity for the hormone of interest. This means an antibody which binds to the hormone of interest under high stringency conditions. For example, the first antibody may be an antibody to luteinizing hormone.

The first antibody may be immobilized on walls of the capture chamber 28, or the first antibody may be immobilized on a substrate 46 that resides in the capture chamber 28. The substrate 46 may be a porous nonreactive carrier matrix. Such matrices are commonly used for nucleic acid and protein binding. Examples of materials suitable for the substrate 46 include nitrocellulose and nylon. When the substrate 46 is nitrocellulose, antibodies can be directly immobilized onto the substrate 46. For other types of substrates 46, immobilization may be accomplished by techniques such as treatment with cyanogen bromide and carbonyldiimidazole known to those skilled in the art. The substrate 46 may be blocked with a blocking agent prior to immobilization of an antibody. Blocking agents may include bovine serum albumin, diluted serum, non-fat dry milk, and casein. Blocking agents prevent binding of unwanted compounds to the substrate 46 or walls of the capture chamber 28, as the case may be.

The device 10 may also include a mixing chamber 49 in fluid communication with the sample transmission tube 31. The mixing chamber 49 provides an area within which the sample and the first reagent can mix.

The device 10 may also include a filter 52. The filter 52 may have an inlet in fluid communication with the sample receiving cup 16 and an outlet in fluid communication with the capture chamber 28. One possible location for the filter 52 is in the mixing chamber. The filter 52 may be designed to remove cells and particulate matter from a whole blood sample, allowing blood serum to pass through the filter 52. In such an embodiment of the invention, the analyte of interest is too small to be removed by the filter 52 and, therefore, passes through the filter 52. The filter 52 may be made from a glass fiber.

The invention may also include a second variable volume reagent container 55 in fluid communication with the capture chamber 28. To provide fluid communication with the capture chamber 28, a second reagent transmission tube 58, which is in fluid communication with the capture chamber 28, may be joined to or capable of joining to the second variable volume reagent container 55. An end 61 of the second reagent transmission tube 58 is beveled to provide a penetrating edge proximate to the second variable volume reagent container 55. The beveled end 61 may be used to pierce the second variable volume reagent container 55 to allow reagent inside to flow from the second variable volume reagent container 55 to the second reagent transmission tube 58. For example, the second variable volume reagent container 55 may be moved toward the beveled end 61 such that a portion of the second variable volume reagent container 55 is pierced by the beveled end 61. The second variable volume reagent container 55 may be collapsible.

The second variable volume reagent container 55 may be collapsible. The second variable volume reagent container 55 may be made from polyethylene, polypropylene, or similar polymers, and may have wall thicknesses small enough to facilitate the collapsible feature. A second plunger 63 may be used to collapse the second variable volume reagent container 55 toward the end 61.

The reagent in the second variable volume reagent container 55 may have a labeled second antibody. The reagent in the second variable volume reagent container 55 may provide additional liquid to assist the propagation of the sample, or a portion of the sample such as blood serum, through the capture chamber 28.

The device 10 may include a reservoir 64 in fluid communication with the capture chamber 28. The reservoir 64 may be used to hold fluids leaving the capture chamber 28. To facilitate holding such fluids, an absorbent material 67, such as paper or cotton fiber or a polymeric fiber with absorbent properties, may be disposed in the reservoir 64. The reservoir 64 may be provided with an exit orifice 70 to allow gas to escape. In order to prevent fluids from leaving the reservoir 64, but allow gas to escape, a hydrophobic material 73 may be disposed in the exit orifice 70.

One or more of the antibodies may be labeled in order to provide for easy detection of the analyte. The antibodies may be labeled with a fluorescent label, a radiolabel, or a chromophore. Examples of labels useful in the invention are radiolabels such as ¹³¹I, ¹¹¹In, ¹²³I, ⁹⁹mTc, ³²p, ¹²⁵I, ³H, ¹⁴C, and ¹⁸⁸Rh, fluorescent labels such as fluorescein and rhodamine, chemiluminescers such as lucifein, enzymatic markers such as peroxidase or phosphatase, and chromophores. A UV photoreactive dye may be used to label an antibody. Procedures for labeling antibodies with labels of these types are described in Wensel et al., Radioimmunoimaging and Radioimmunotherepy, Elsevier, N.Y. (1983); Colcher et al., Meth. Enzymol., 121: 802-16 (1986); Goding et al., Monoclonal Antibodies: Principles and Practice, pp. 124-26 (1983) (“Goding”); Hunter et al., Nature, 144:945 (1962); David et al., Biochemistry, 13:1014-21 (1974); Grenwood et al., Biochem J., 89:114-23 (1963); Marchalonis, Biochem J., 113:299-305 (1969); and Morrison et al., Immunochemistry, 289-297 (1971). Suitable chromophores are described by Stryker, Science, 162:526 (1968) and Brand et al., Ann Review of Biochem., 41:843-68 (1972).

The antibodies may be monoclonal or polyclonal. The particular choice of antibody will depend on the analyte of interest. For example, for detection of luteinizing hormone (“LH”), antibodies which have specific binding affinity to LH may be used. The antibodies may be supplied by antibody suppliers known to those skilled in the art. Alternatively, monoclonal antibody production may be effected by techniques well-known in the art.

Instead of including the second antibody in the second variable volume reagent container 55, the second antibody may be included in the first variable volume reagent container 13 or in both variable volume reagent containers 13, 55. In addition, a second antibody may be included in the first variable volume reagent container 13 and a third antibody may be included in the second variable volume reagent container 55. When more than one antibody is used in the detector 10, the first antibody may be specific to the combination of the analyte of interest: and the other antibody, or antibodies as the case may be.

In addition to utilizing whole antibodies, the invention includes use of binding portions of such antibodies. It should be noted that the term “antibodies” is used herein to include both whole antibodies and binding portions of antibodies. Such binding portions include Fab fragments, F(ab′)₂ fragments, and Fv fragments. These antibody fragments can be made by conventional procedures, such as proteolytic fragmentation procedures as described in Goding, Monoclonal Antibodies: Principles and Practice, pp. 98-118 (N.Y. Academic Press 1983).

The capture chamber 28 may include a translucent material, such as acrylic, that facilitates transmission of electromagnetic waves. For example, where the label is a chromophore, the color produced by the chromophore may be measured by comparison to a color chart to determine the LH Level. In addition, various alternative means can be employed for visually reading the capture chamber 28. This would include means for assessing color differences from a solid surface. One illustrative embodiment is described in U.S. patent application Ser. No. 09/208,648.

The invention includes an analyte detection method. One embodiment of the method is illustrated in FIG. 5. In the method, an analyte detector, such as that described above, is provided (operation 100). A sample of body fluid is provided (operation 103) in the sample receiving cup. For example, the sample of body fluid may be blood. The sample may be allowed to flow into a tube connecting the sample receiving cup and the first variable volume reagent container. A plug may be provided (operation 106) in the sample receiving cup to prevent the sample of body fluid from re-entering the sample receiving cup.

The volume of the first variable volume reagent container is reduced (operation 109) to force the first reagent toward the capture chamber. For example, the volume may be reduced (operation 109) by compressing the first variable volume reagent container to collapse the container. The first variable volume reagent container may be pierced to allow reagent therein to move toward the sample of body fluid.

The sample may be filtered (operation 112) to remove particulate matter. For example, when the sample is blood, the sample may be filtered to remove cellular matter and cellular debris, and similar particulate matter.

Once the sample and the first reagent move into the capture chamber, the first antibody combines with the analyte of interest that is in the sample. The capture chamber is then analyzed (operation 115) to determine whether the first antibody combined with the analyte of interest. For example, the capture chamber may be analyzed (operation 115) to detect electromagnetic radiation, such as light reflected or produced by a labeled antibody, emanating from the capture chamber.

The invention is particularly useful because it does not require extensive training to obtain accurate results. The invention may be used by breeders, such as swine breeders, who use artificial insemination (“AI”) techniques to promote conceptions. AI is an expensive procedure, costing nearly $19 per attempt. In contrast, monoclonal antibody blood testing as described herein is estimated to cost only about $2 per test, a savings of nearly 90%. Therefore, the invention could be used in conjunction with AI techniques to maximize the advantages of the latter, eliminating expensive, failed inseminations. Thus, the invention will save the animal breeding industry substantial amounts of money. For example, in 1998 it: is estimated that LH testing will save swine AI breeders $252 million; 2002, it is predicted that these savings will reach $410 million.

The invention also has zoological significance. In order to ensure a diverse gene pool, captive species are often mated with animals from other zoos. However, this procedure is complicated and expensive. Therefore, particularly with large animals, it is important that the female is fertile at the time of insemination or mating. The method of the invention, therefore, will be a useful aid in determining the fertility of the mammal, aiding in successful impregnation at reduced cost.

Although the invention has been described in detail for the purpose of illustration, it is understood that such detail is solely for illustration and variations can be made by those skilled in the art without departing from the spirit and scope of the invention, which is defined by the following claims.

What is claimed is:

1. An analyte detector, comprising:
a first variable volume reagent container;
a sample receiving cup in fluid communication with the first variable volume reagent container;
a capture chamber in fluid communication with the first variable volume reagent container and the sample receiving cup; and
a first antibody residing in the capture chamber, the first antibody being specific for an analyte of interest.

2. The analyte detector of claim 1, wherein the first variable volume reagent container is collapsible.

3. The analyte detector of claim 1, further comprising a first reagent transmission tube joined to the first variable volume reagent container and joined to the sample receiving cup.

4. The analyte detector of claim 3, wherein at least part of the first reagent transmission tube is a capillary tube.

5. The analyte detector of claim 3, wherein an end of the first reagent transmission tube is beveled to provide a penetrating edge proximate to the first variable volume reagent container.

6. The analyte detector of claim 1, further comprising a sample transmission tube joined to the sample receiving cup and joined to the capture chamber.

7. The analyte detector of claim 6, wherein at least part of the sample transmission tube is a capillary tube.

8. The analyte detector of claim 6, further comprising a first reagent transmission tube joined to the first variable volume reagent container and joined to the sample receiving cup, and further comprising a drain joining the sample cup to the first reagent transmission tube and the sample transmission tube.

9. The analyte detector of claim 1, further comprising a plug having a diameter approximately equal to a diameter of the drain.

10. The analyte detector of claim 1, further comprising a mixing chamber in fluid communication with the sample transmission tube.

11. The analyte detector of claim 10, further comprising a filter disposed in the mixing chamber.

12. The analyte detector of claim 1, further comprising a filter having an inlet in fluid communication with the sample cup and an outlet in fluid communication with the capture chamber.

13. The analyte detector of claim 1, further comprising a second variable volume reagent container in fluid communication with the capture chamber.

14. The analyte detector of claim 13, wherein the second variable volume reagent container is collapsible.

15. The analyte detector of claim 13, further comprising a second reagent transmission tube joined to the second variable volume reagent container and in fluid communication with the capture chamber.

16. The analyte detector of claim 15, wherein an end of the second reagent transmission tube is beveled to provide a penetrating edge proximate to the second variable volume reagent container.

17. The analyte detector of claim 1, further comprising a reservoir in fluid communication with the capture chamber.

18. The analyte detector of claim 17, further comprising an absorbent material disposed in the reservoir.

19. The analyte detector of claim 18, wherein the reservoir has an exit orifice.

20. The analyte detector of claim 19, further comprising a hydrophobic material disposed in the exit orifice.

21. The analyte detector of claim 1, wherein the first antibody is labeled.

22. The analyte detector of claim 1, wherein the first antibody is labeled with a label selected from the group consisting of a fluorescent label, a radiolabel, and a chromophore.

23. The analyte detector of claim 1, wherein the analyte of interest is a mammallian hormone.

24. The analyte detector of claim 23, wherein the mammal hormone is selected from the group consisting of luteinizing hormone, estradiol, follicle-stimulating hormone, progesterone, and combinations thereof.

25. The analyte detector of claim 1, wherein the first antibody is a monoclonal antibody.

26. The analyte detector of claim 1, wherein the first antibody is a polyclonal antibody.

27. The analyte detector of claim 1, wherein the first antibody is immobilized in the capture chamber.

28. The analyte detector of claim 1, wherein a second antibody is in the first variable volume reagent container.

29. The analyte detector of claim 1, further comprising a second antibody specific to a combination of the analyte of interest and the first antibody.

30. The analyte detector of claim 29, wherein the second antibody is labeled.

31. The analyte detector of claim 29, wherein the second antibody is labeled with a label selected from the group consisting of a fluorescent label, a radiolabel, and a chromophore.

32. The analyte detector of claim 29, wherein the second antibody is in the second variable volume reagent container.

33. An analyte detection method, comprising:
providing an analyte detector having a first variable volume reagent container with a first reagent therein, and having a sample receiving cup in fluid communication with the first variable volume reagent container, and having a capture chamber in fluid communication with the first variable volume reagent container and the sample receiving cup, and having a first antibody residing in the capture chamber, the first antibody being specific for an analyte of interest;
providing a sample of body fluid in the sample receiving cup;
reducing the volume of the first variable volume reagent container to force the first reagent toward the capture chamber;
determining whether the first antibody combined with the analyte of interest.

34. The analyte detection method of claim 33, wherein the sample of body fluid is blood.

35. The analyte detection method of claim 33, wherein reducing the volume of the first variable volume reagent container includes compressing the first variable volume reagent container.

36. The analyte detection method of claim 33, further comprising piercing the first variable volume reagent container to release the first reagent from the first variable volume reagent container.

37. The analyte detection method of claim 33, further comprising providing a plug in the sample receiving cup.

38. The analyte detection method of claim 33, wherein determining whether the first antibody combined with the analyte of interest includes detecting electromagnetic radiation emanating from the capture chamber.

39. The analyte detection method of claim 33, wherein the analyte of interest is selected from the group consisting of luteinizing hormone, estradiol, follicle-stimulating hormone, progesterone, and combinations thereof.

40. The analyte detection method of claim 33, further comprising filtering the sample of body fluid.
